(19) United States
(12) Patent Application Publication
(10) Pub. No.: US 2011/0124542 A1
(43) Pub. Date: May 26, 2011

(54) Composition Containing Surfactants and Having a Special Emulsifier Mixture

(76) Inventor: Kirsten Sartingen, Brueggen (DE)

(21) Appl. No.: 13/010,248

(22) Filed: Jan. 20, 2011

Related U.S. Application Data
(63) Continuation of application No. PCT/EP2009/058665, filed on Jul. 8, 2009.

(30) Foreign Application Priority Data
Jul. 23, 2008 (DE) 10 2008 034 388.9

(51) Int. Cl. A61K 8/37; A61Q 5/02; A61Q 19/00; C11D 17/00; A61Q 19/10

(52) U.S. Cl. 510/119; 510/130; 510/137; 510/138; 510/158; 510/506

(57) ABSTRACT

Clear composition comprising surfactants, one or more oil component(s), and a mixture of solubilizers chosen from (a) ethoxylated fatty alcohols, (b) ethoxylated hydrogenated castor oils, and (c) ethoxylated mono-, di- or triglycerol esters in a ratio of components (a):(b):(c) in the range of 1:(2-4):(3-4). The compositions provide a particular suspension stability and viscosity stability and are suitable as a cosmetic composition for cleaning skin and hair.

### CROSS REFERENCE TO RELATED APPLICATIONS

The present application is a continuation of International Patent Application No. PCT/EP2009/058665 filed 8 Jul. 2009, which claims priority to German Patent Application No. 10 2008 034 388.9 filed 23 Jul. 2008, both of which are incorporated herein by reference.

The present invention relates to a composition based on surfactants in which one or more oil components are stably suspended by means of a special emulsifier mixture, as well as to the use of the composition as a cosmetic agent.

Cleaning agents such as cosmetic cleaning agents for the skin and hair (e.g., liquid soaps, shampoos, shower washes, foam baths, shower and washing gels) not only have to have good cleaning ability but also must be well tolerated by the skin and mucosa and must not lead to severe degreasing or skin dryness, even when used frequently.

Therefore, attempts have been made over the years to incorporate as many hair and skin conditioning agents as possible into the cleaning agents. This is to satisfy requirements for simultaneous cleaning and care, as well as allowing the consumer to enjoy both the benefit of time savings and cost savings through use of just one product that meets both of these requirements (as a care stage, using a supplementary skin or hair treatment agent should ideally become superfluous).

However, incorporation of skin and hair conditioning agents into a surfactant-based cleaning formulation continues to present manufacturers of such compositions with serious problems, as mineral, natural or synthetic fat, wax and oil components that provide a lasting caring and conditioning effect on the skin and hair cannot be easily incorporated into a surfactant base by simple mixing.

Thus, in the past it was necessary, for example, to incorporate pearlescent agents, special polymers and/or salts into the cosmetic cleaning agents in order to be able to stably suspend, for example, silicones in the formulations.

The trend is now returning to natural cosmetic products based on mild (where possible, biodegradable) surfactants containing natural conditioning agents of plant origin. In these products, those active ingredients which can potentially irritate the skin or scalp and which are not absolutely necessary are preferably kept to a minimum, and even with required components it is very important to consider skin tolerability, mildness and biodegradability.

This gave rise to a completely new problem, since stabilizing a natural oil component in a mild surfactant base using as few chemical auxiliary substances as possible is not a trivial matter. For one thing it was not always possible to produce clear formulations (desired by the consumer as they are associated with natural compositions containing no “chemicals”). Also, incorporation of natural oils into the surfactant base was usually associated with major problems regarding their suspension stability over an extended period of time and in varying temperatures and with a dramatic drop in the viscosity of the compositions.

The present invention therefore provides a clear surfactant-based cleaning agent wherein an oil component (ideally a plant-based oil component) is stably suspended over a long period of time and in varying temperatures. The invention also provides a clear cleaning agent having a sufficiently high viscosity to enable it to be easily applied from the container without “running” and dripping. This initial viscosity should as far as possible be retained during storage.

These were achieved to a great extent by identifying a special emulsifier mix which has to be present in the surfactant base in a specific mixing ratio. Using this emulsifier mixture it was possible to incorporate a large number of oil components stably into a (mild) surfactant base, and it was found that even when stored at high temperatures (50 or 60° C.), the suspension action was retained and the viscosity of the composition did not change.

It was possible to use the emulsifier mixture in all common surfactant systems; in particular, it was possible to choose a mild surfactant base and plant-based oils and still achieve the clear surfactant-based cleaning agent according to the invention. Clear compositions according to the invention are thus suitable as cosmetic cleaning agents for skin and hair as they satisfy the previously mentioned needs of consumers for mild, natural products.

The present invention therefore provides a clear composition containing surfactants, one or more oil component(s), and a mixture of solubilizers chosen from
a) ethoxylated fatty alcohols,
b) ethoxylated hydrogenated castor oils, and
c) ethoxylated mono-, di- or triglycerol esters,
   wherein the ratio of components a):b):c) is in the range of 1:(2-4):(3-4).

According to the invention a clear composition is understood to be a composition having an NTU value (nephelometric turbidity unit) of ≦50, preferably ≦30, particularly preferably 20 and in particular ≦10.

Component a) of the emulsifier mixture according to the invention is an ethoxylated fatty alcohol, which can be a straight-chain or branched, saturated or unsaturated C₈-C₂₂ fatty alcohol, preferably a C₁₀-C₂₀ fatty alcohol, particularly preferably a C₈-C₂₂ fatty alcohol and in particular a C₁₃-C₁₅ fatty alcohol having a degree of ethoxylation of 1 to 18, preferably 5 to 16, particularly preferably 7 to 14 and in particular 8 to 12.

Preferred components a) according to the invention include deceth-5, deceth-7, undeceth-7, laureth-4, laureth-5, laureth-6, laureth-7, laureth-8, laureth-9, laureth-10, laureth-12, trideceth-5, trideceth-7, trideceth-8, trideceth-9, trideceth-10, trideceth-12, myreth-8, myreth-10 and myreth-12. Laureth-7, laureth-8, laureth-9, laureth-10, trideceth-5, trideceth-7, trideceth-8, trideceth-9 and trideceth-10 are particularly preferred, with trideceth-9 being even more particularly preferred.

According to the invention component b) is ethoxylated hydrogenated castor oil having a degree of ethoxylation of 5 to 80, preferably 10 to 60, particularly preferably 25 to 50 and in particular 35 to 45.

PEG-10 hydrogenated castor oil, PEG-25 hydrogenated castor oil, PEG-40 hydrogenated castor oil and PEG-60 hydrogenated castor oil are suitable, with PEG-40 hydrogenated castor oil being particularly suitable, such as is available commercially under the trade names Cremophor® RH 410 or Eumulgin® HRE 455.

It is also possible according to the invention to use components a) and b) in a mixture. Such solubilizer mixtures are available commercially for example under the name Solubilizer® 611674 or Solubilizer® 660352.

Component c) of the emulsifier mixture according to the invention is a monoester and/or a mixture of monoesters and diesters of glycerol with branched or straight-chain, saturated or unsaturated fatty acids having a C chain length of 8 to 24, preferably 10 to 18 and in particular 12 to 16, which have a degree of ethoxylation of 1 to 20, preferably 2 to 17, particularly preferably 4 to 13 and in particular 6 to 10.

Ethoxylated glyceryl oleates and glyceryl cocoates are preferred according to the invention, and PEG-7 glyceryl cocoate is particularly preferred, such as is commercially available under the name Tegosoft® GC or Cetiol® HE.

The three emulsifiers a), b) and c) are each used in compositions according to the invention in an amount of 0.05 to 3 wt. %, preferably 0.1 to 2 wt. % and in particular 0.2 to 1.5 wt. %, based on total weight of the composition.

In order to satisfy viscosity stability, compositions according to the invention have a viscosity in the range from 5000 to 9500 mPas, preferably from 6000 to 9000 mPas and in particular from 7000 to 8000 mPas (measured in each case using a Haake rotary viscometer VT550; 20° C.; measuring device MV; spindle MV II; 8 rpm). Compositions with this viscosity can be conveniently and easily applied from a container onto the hand or application surface without running through the fingers and dripping. At the same time, the viscosity of the composition is low enough to ensure that it can be satisfactorily distributed over the application surface using the hands.

The pH of compositions according to the invention is ideally within a range that is gentle on the skin such as around 3 to 7, preferably in a range from 4 to 6.5 and particularly in a range from 4.5 to 5.5.

A further substantial constituent of compositions according to the invention is one or more oil component(s).

These can be chosen from mineral, natural or synthetic oil components such as petroleum jelly, paraffins, silicones, fatty alcohols, fatty acids, fatty acid esters and natural oils of plant and animal origin, and are used in the compositions in an amount of from 0.005 to 20 wt. %, preferably 0.01 to 10 wt. %, more preferably 0.05 to 5 wt. % and particularly 0.2 to 3 wt. %, based on total weight of the composition.

The term “silicone oils” is understood by the person skilled in the art to mean various structures of organosilicon compounds.

Silicones are preferably chosen from at least one representative of organosilicon compounds formed from:
(i) polyalkylsiloxanes, polyarylsiloxanes, and polyalkylarylsiloxanes, which are volatile or non-volatile, straight-chain, branched or cyclic, crosslinked or non-crosslinked;
(ii) polysiloxanes, which in their general structure contain one or more organofunctional groups and are chosen from:
   a) substituted or unsubstituted aminated groups;
   b) (per)fluorinated groups;
   c) thiol groups;
   d) carboxylate groups;
   e) hydroxylated groups;
   alkoxylated groups;
   g) acyloxyalkyl groups;
   h) amphoteric groups;
   i) bisulfite groups;
   j) hydroxyacyl amino groups;
   k) carboxy groups
   l) sulfonic acid groups; and
   m) sulfate or thiosulfate groups;
(iii) linear polysiloxane(A)-polyoxyalkylene(B) block copolymers of the type (A-B)ₙ, where n>3;
(iv) grafted silicone polymers having a non-silicone-containing, organic framework consisting of an organic main chain formed from organic monomers containing no silicone, onto which at least one polysiloxane macromer has been grafted in the chain and optionally on at least one chain end;
(v) grafted silicone polymers having a polysiloxane framework onto which non-silicone-containing, organic monomers have been grafted, which have a polysiloxane main chain onto which at least one organic macromer containing no silicone has been grafted in the chain and optionally on at least one of its ends, such as the commercial product Abil B 8832 from Degussa sold under the INCI designation Bis-PEG/PPG-20/20 Dimethicone;
(vi) or mixtures thereof.

In one embodiment of the present invention, the conditioning agent is a conditioning silicone having a viscosity of 20,000 to 120,000 mPa·s, preferably 40,000 to 80,000 mPa·s.

The conditioning silicone is preferably chosen from dimethicones, amodimethicones or dimethiconols.

Linear and/or branched, saturated and/or unsaturated fatty acids having 6 to 30 carbon atoms can be used as fatty acids. Fatty acids having 10 to 22 carbon atoms are preferred. Examples include isostearic acids such as the commercial products Emersol® 871 and Emersol® 875, and isopalmitic acids such as the commercial product Edenor® IP 95, as well as all other fatty acids sold under the Edenor® trade names (Cognis). Further typical examples of such fatty acids are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachidic acid, gadoleic acid, behenic acid and erucic acid and technical mixtures thereof, which form for example in the pressurized cleavage of natural fats and oils, in the oxidation of aldehydes from the Roelen oxo synthesis or the dimerization of unsaturated fatty acids. Fatty acid cuts obtainable from coconut oil or palm oil are conventionally particularly preferred; use of stearic acid is also particularly preferred.

The amount used here is 0.1 to 15 wt. %, based on total weight of the agent. The amount is preferably 0.5 to 10 wt. %, wherein amounts of 1 to 5 wt. % can be most particularly advantageous.

Saturated, mono- or polyunsaturated, branched or unbranched fatty alcohols having C₆ to C₃₀, preferably C₁₀ to C₂₂ and most particularly preferably C₁₂ to C₂₂ carbon atoms can be used as fatty alcohols. Suitable for use are decanol, octanol, octenol, dodecenol, decenol, octadienol, dodecadienol, decadienol, oleyl alcohol, erucic alcohol, ricinol alcohol, stearyl alcohol, isostearyl alcohol, cetyl alcohol, lauryl alcohol, myristyl alcohol, arachidyl alcohol, capryl alcohol, capric alcohol, linoleyl alcohol, linolenyl alcohol and behenyl alcohol, as well as the Guerbet alcohols thereof, wherein this list is intended to be of an exemplary and non-limiting nature. However, fatty alcohols derive from preferably natural fatty acids, wherein it can conventionally be assumed that they are obtained from esters of fatty acids by reduction. Suitable for use according to the invention are likewise such fatty alcohol cuts produced by reduction of naturally occurring triglycerides such as beef fat, palm oil, groundnut oil, colza oil, cottonseed oil, soybean oil, sunflower oil and linseed oil or from fatty acid esters formed from the transesterification products thereof with corresponding alcohols, and which thus represent a mixture of different fatty alcohols. Such substances are commercially available, for example, under the names Stenol® (e.g., Stenol® 1618), Lanette® (e.g., Lanette® O), or Lorol® (e.g., Lorol® C8, Lorol® C14, Lorol® C18, Lorol® C8-18), HD-Ocenol®, Crodacol® (e.g., Crodacol® CS), Novol®, Eutanol® G, Guerbitol® 16, Guerbitol® 18, Guerbitol® 20, Isofol® 12, Isofol® 16, Isofol® 24, Isofol® 36, Isocarb® 12, Isocarb® 16 or Isocarb® 24. Wool wax alcohols can of course also be used according to the invention, such as are commercially available under the names Corona®, White Swan®, Coronet® or Fluilan®. Fatty alcohols are used in amounts of 0.1 to 30 wt. %, preferably in amounts of 0.1 to 20 wt. %, based on total weight of the preparation.

Solid paraffins or isoparaffins, carnauba waxes, beeswaxes, candelilla waxes, ozokerites, ceresin, spermaceti wax, sunflower wax, fruit waxes such as apple wax or citrus wax, and PE or PP microwaxes can be used according to the invention as natural or synthetic waxes. Such waxes are available, for example, via Kahl & Co., Trittau.

The amount used is 0.1 to 50 wt. %, relative to the complete agent, preferably 0.1 to 20 wt. % and particularly preferably 0.1 to 15 wt. %, relative to the complete agent.

Natural and synthetic cosmetic oil bodies include:
liquid paraffin oils, isoparaffin oils and synthetic hydrocarbons and also di-n-alkyl ethers having in total 12 to 36 C atoms, particularly 12 to 24 C atoms, such as di-n-octyl ether, di-n-decyl ether, di-n-nonyl ether, di-n-undecyl ether, di-n-dodecyl ether, n-hexyl-n-octyl ether, n-octyl-n-decyl ether, n-decyl-n-undecyl ether, n-undecyl-n-dodecyl ether and n-hexyl-n-undecyl ether and also di-tert-butyl ether, diisopentyl ether, di-3-ethyl decyl ether, tert-butyl-n-octyl ether, isopentyl-n-octyl ether and 2-methyl pentyl-n-octyl ether. Commercially available compounds 1,3-di-(2-ethylhexyl)cyclohexane (Cetiol® S) and di-n-octyl ether (Cetiol® OE) can be preferred.
ester oils. Ester oils refer to the esters of C₆-C₃₀ fatty acids with C₂-C₃₀ fatty alcohols. The monoesters of fatty acids with alcohols having 2 to 24 C atoms are preferred. Examples of fatty acid components used in the esters are caproic acid, caprylic acid, 2-ethylhexanoic acid, capric acid, lauric acid, isotridecanoic acid, myristic acid, palmitic acid, palmitoleic acid, stearic acid, isostearic acid, oleic acid, elaidic acid, petroselic acid, linoleic acid, linolenic acid, elaeostearic acid, arachidic acid, gadoleic acid, behenic acid and erucic acid and technical mixtures thereof, which form, for example, in the pressurized cleavage of natural fats and oils, in the oxidation of aldehydes from Roelen oxo synthesis or dimerization of unsaturated fatty acids. Examples of fatty alcohol components in the ester oils are isopropyl alcohol, hexanol, octanol, 2-ethylhexyl alcohol, decanol, lauryl alcohol, isotridecyl alcohol, myristyl alcohol, cetyl alcohol, palmoleyl alcohol, stearyl alcohol, isostearyl alcohol, oleyl alcohol, elaidyl alcohol, petroselinyl alcohol, linolyl alcohol, linolenyl alcohol, elaeostearyl alcohol, arachyl alcohol, gadoleyl alcohol, behenyl alcohol, erucyl alcohol and brassidyl alcohol and the technical mixtures thereof which form, for example, in the high-pressure hydrogenation of technical methyl esters based on fats and oils or aldehydes from Roelen oxo synthesis and as a monomer fraction in dimerization of unsaturated fatty alcohols. Particularly preferred according to the invention are isopropyl myristate (Rilanit® IPM), isononanoic acid C₁₆₋₁₈ alkyl ester (Cetiol® SN), 2-ethylhexyl palmitate (Cegesoft® 24), stearic acid 2-ethylhexyl ester (Cetiol® 868), cetyl oleate, glycerol tricaprylate, coconut fatty alcohol caprinate/caprylate (Cetiol® LC), n-butyl stearate, oleyl erucate (Cetiol® J 600), isopropyl palmitate (Rilanit® IPP), oleyl oleate (Cetiol®), lauric acid hexyl ester (Cetiol® A), di-n-butyl adipate (Cetiol® B), myristyl myristate (Cetiol® MM), cetearyl isononanoate (Cetiol® SN), oleic acid decyl ester (Cetiol® V).
dicarboxylic acid esters such as di-n-butyl adipate, di-(2-ethylhexyl) adipate, di-(2-ethylhexyl) succinate and diisotridecyl acelate and also diol esters such as ethylene glycol dioleate, ethylene glycol diisotridecanoate, propylene glycol di-(2-ethyl hexanoate), propylene glycol diisostearate, propylene glycol dipelargonate, butanediol diisostearate, neopentyl glycol dicaprylate,
symmetrical, asymmetrical or cyclic esters of carbonic acid with fatty alcohols, as described for example in DE-OS 197 56 454, glycerol carbonate or dicaprylyl carbonate (Cetiol® CC),
tri-fatty acid esters of saturated and/or unsaturated linear and/or branched fatty acids with glycerol,
fatty acid partial glycerides, namely monoglycerides, diglycerides and technical mixtures thereof. If technical products are used, small amounts of triglycerides can also be included for production reasons. Partial glycerides preferably are of the formula

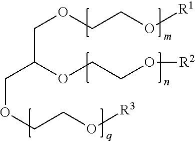

wherein R′, R² and R³ are each independently hydrogen or a linear or branched, saturated and/or unsaturated acyl residue having 6 to 22, preferably 12 to 18, carbon atoms, with the proviso that at least one of these groups is an acyl residue and at least one of these groups is hydrogen. The sum (m+n+q) is 0 or a number from 1 to 100, preferably 0 or 5 to 25. R′ preferably is an acyl residue and R² and R³ hydrogen, and the sum (m+n+q) is preferably 0. Typical examples are mono- and/or diglycerides based on caproic acid, caprylic acid, 2-ethylhexanoic acid, capric acid, lauric acid, isotridecanoic acid, myristic acid, palmitic acid, palmoleic acid, stearic acid, isostearic acid, oleic acid, elaidic acid, petroselic acid, linoleic acid, linolenic acid, elaeostearic acid, arachidic acid, gadoleic acid, behenic acid and erucic acid and technical mixtures thereof. Oleic acid monoglycerides are preferably used.

In a particularly preferred embodiment of the invention plant-based oil is used as the oil component.

Suitable natural oils include amaranth seed oil, apricot kernel oil, argan oil, avocado oil, babassu oil, cottonseed oil, borage seed oil, camelina oil, thistle oil, groundnut oil, pomegranate kernel oil, grapefruit seed oil, hemp oil, rosehip seed oil, hazelnut oil, elderberry seed oil, blackcurrant seed oil, jojoba oil, cocoa butter, linseed oil, macadamia nut oil, maize oil, almond oil, manila oil, evening primrose oil, olive oil, palm oil, peach kernel oil, rapeseed oil, rice oil, sea buckthorn fruit oil, sea buckthorn seed oil, sesame oil, shea butter, soybean oil, sunflower oil, grape seed oil, walnut oil or wild rose oil. Avocado oil, apricot kernel oil, rosehip seed oil, jojoba oil, cocoa butter, almond oil, olive oil, peach kernel oil, shea butter, sunflower oil and grape seed oil are particularly preferred according to the invention.

Plant-based oils are used in agents according to the invention in an amount of 0.001 to 10 wt. %, preferably 0.005 to 7 wt. %, particularly preferably 0.01 to 5 wt. % and in particular 0.05 to 3 wt. %, based on total weight of the agent.

In addition to the emulsifier mixture and the oil component, the surfactant base is the third component of the composition according to the invention.

The term “surfactants” refers to surface-active substances having an anionic or a cationic charge, or an anionic and a cationic charge, in the molecule. Surface-active substances can also be non-ionic.

All anionic surface-active substances suitable for use on the human body are suitable as anionic surfactants in preparations according to the invention. These have the characterizing feature of a water-solubilizing anionic group such as a carboxylate, sulfate, sulfonate or phosphate group and a lipophilic alkyl group having approximately 8 to 30 C atoms. The molecule can additionally contain glycol or polyglycol ether groups, ester, ether and amide groups and hydroxyl groups. Examples of suitable anionic surfactants, each in the form of the sodium, potassium and ammonium salts as well as the mono-, di- and trialkanolammonium salts having 2 to 4 C atoms in the alkanol group, are—
linear and branched fatty acids having 8 to 30 C atoms (soaps),
ether carboxylic acids of the formula R—O—(CH₂—CH₂O)ₓ—CH₂—COOH, wherein R is a linear alkyl group having 8 to 30 C atoms and x=0 or 1 to 16,
acyl sarcosides having 8 to 24 C atoms in the acyl group,
acyl taurides having 8 to 24 C atoms in the acyl group,
acyl isethionates having 8 to 24 C atoms in the acyl group,
sulfosuccinic acid mono- and dialkyl esters having 8 to 24 C atoms in the alkyl group and sulfosuccinic acid monoalkyl polyoxyethyl esters having 8 to 24 C atoms in the alkyl group and 1 to 6 oxyethyl groups,
linear alkane sulfonates having 8 to 24 C atoms,
linear alpha-olefin sulfonates having 8 to 24 C atoms,
alpha-sulfo fatty acid methyl esters of fatty acids having 8 to 30 C atoms,
alkyl sulfates and alkyl polyglycol ether sulfates of formula R—O (CH₂—CH₂O)ₓOSO₃H, wherein R is a preferably linear alkyl group having 8 to 30 C atoms and x=0 or 1 to 12,
mixtures of surface-active hydroxy sulfonates according to DE-A-37 25 030,
sulfated hydroxyalkyl polyethylene and/or hydroxyalkylene propylene glycol ethers according to DE-A-37 23 354,
sulfonates of unsaturated fatty acids having 8 to 24 C atoms and 1 to 6 double bonds according to DE-A-39 26 344,
esters of tartaric acid and citric acid with alcohols which are addition products of around 2 to 15 molecules of ethylene oxide and/or propylene oxide with fatty alcohols having 8 to 22 C atoms,
alkyl and/or alkenyl ether phosphates of formula (TI)—

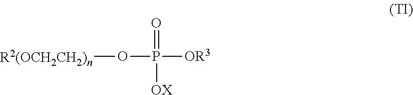

wherein R²⁹ is preferably an aliphatic hydrocarbon residue having 8 to 30 carbon atoms, R³⁰ is hydrogen, a (CH₂CH₂O)ₙR²⁹ residue or X, n is a number from 1 to 10, and X is hydrogen, an alkali or alkaline-earth metal or NR³¹R³²R³³R³⁴, where R³¹ to R³⁴ independently of one another is a C₁ to C₄ hydrocarbon residue,
sulfated fatty acid alkylene glycol esters of the formula (TII)

R³⁵CO(AlkO)ₙSO₃M (TII)
wherein R³⁵CO is a linear or branched, aliphatic, saturated and/or unsaturated acyl residue having 6 to 22 C atoms, Alk is CH₂CH₂, CHCH₃CH₂ and/or CH₂CHCH₃, n is a number from 0.5 to 5 and M is a cation such as described in DE-OS 197 36 906.5,
monoglyceride sulfates and monoglyceride ether sulfates of formula (TIII)—

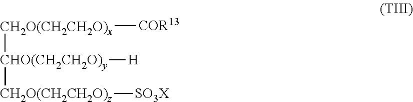

wherein R¹³CO is a linear or branched acyl residue having 6 to 22 carbon atoms, x, y and z in total is 0 or a number from 1 to 30, preferably 2 to 10, and X is an alkali or alkaline-earth metal. Typical examples of suitable monoglyceride (ether) sulfates within the meaning of the invention are transesterification products of lauric acid monoglyceride, coconut fatty acid monoglyceride, palmitic acid monoglyceride, stearic acid monoglyceride, oleic acid monoglyceride and tallow fatty acid monoglyceride as well as the ethylene oxide adducts thereof with sulfur trioxide or chlorosulfonic acid in the form of their sodium salts. Monoglyceride sulfates according to formula (TIII) are preferably used wherein R³⁶CO is a linear acyl residue having 8 to 18 carbon atoms.

Preferred anionic surfactants are alkyl sulfates, alkyl polyglycol ether sulfates and ether carboxylic acids having 10 to 18 C atoms in the alkyl group and up to 12 glycol ether groups in the molecule and sulfosuccinic acid mono- and dialkyl esters having 8 to 18 C atoms in the alkyl group and sulfosuccinic acid monoalkyl polyoxyethyl esters having 8 to 18 C atoms in the alkyl group and 1 to 6 oxyethyl groups.

Surface-active compounds classified as zwitterionic surfactants are those bearing at least one quaternary ammonium group and at least one —COO⁽⁻⁾ or —SO₃⁽⁻⁾ group in the molecule. Particularly suitable zwitterionic surfactants are betaines such as N-alkyl-N,N-dimethylammonium glycinates, for example cocoalkyl dimethylammonium glycinate, N-acyl aminopropyl-N,N-dimethylammonium glycinates, for example cocoacylaminopropyl dimethylammonium glycinate, and 2-alkyl-3-carboxymethyl-3-hydroxyethyl imidazolines, each having 8 to 18 C atoms in the alkyl or acyl group, and cocoacylaminoethyl hydroxyethyl carboxymethyl glycinate. A preferred zwitterionic surfactant is the fatty acid amide derivative known under the INCl name Cocamidopropyl Betaine.

Ampholytic surfactants are understood to be surface-active compounds which, in addition to a C₈-C₂₄ alkyl or acyl group, contain at least one free amino group and at least one —COOH or —SO₃H group in the molecule and are capable of forming internal salts. Examples of suitable ampholytic surfactants are N-alkyl glycines, N-alkyl propionic acids, N-alkyl aminobutyric acids, N-alkyl iminodipropionic acids, N-hydroxyethyl-N-alkyl amidopropyl glycines, N-alkyl taurines, N-alkyl sarcosines, 2-alkyl aminopropionic acids and alkyl aminoacetic acids, each having approximately 8 to 24 C atoms in the alkyl group. Particularly preferred ampholytic surfactants are N-cocoalkyl aminopropionate, cocoacylaminoethyl aminopropionate and C₁₂-C₁₈ acyl sarcosine.

Non-ionic surfactants contain as a hydrophilic group a polyol group, a polyalkylene glycol ether group or a combination of a polyol and polyglycol ether group, for example. Such compounds include—
addition products of 2 to 50 mol of ethylene oxide and/or 0 to 5 mol of propylene oxide with linear and branched fatty alcohols having 8 to 30 C atoms, with fatty acids having 8 to 30 C atoms and with alkyl phenols having 8 to 15 C atoms in the alkyl group,
addition products of 2 to 50 mol of ethylene oxide and/or 0 to 5 mol of propylene oxide with linear and branched fatty alcohols having 8 to 30 C atoms, with fatty acids having 8 to 30 C atoms and with alkyl phenols having 8 to 15 C atoms in the alkyl group, end-capped with a methyl or C₂ to C₆ alkyl residue, such as those available under the commercial names Dehydrol® LS and Dehydrol® LT (Cognis),
C₁₂-C₃₀ fatty acid monoesters and diesters of addition products of 1 to 30 mol of ethylene oxide with glycerol,
addition products of 5 to 60 mol of ethylene oxide with castor oil and hydrogenated castor oil,
polyol fatty acid esters, such as for example the commercial product Hydagen® HSP (Cognis) or Sovermol types (Cognis),
alkoxylated triglycerides,
alkoxylated fatty acid alkyl esters of the formula

R³⁷CO—(OCH₂CHR³⁸)_{w}OR³⁹ (TIV)
wherein R³⁷CO is a linear or branched, saturated and/or unsaturated acyl residue having 6 to 22 carbon atoms, R³⁸ is hydrogen or methyl, R³⁹ is linear or branched alkyl residues having 1 to 4 carbon atoms and w is a number from 1 to 20,
amine oxides,
hydroxy mixed ethers, such as are described in DE-OS 19738866,
sorbitan fatty acid esters and addition products of ethylene oxide with sorbitan fatty acid esters such as polysorbates,
sugar fatty acid esters and addition products of ethylene oxide with sugar fatty acid esters,
addition products of ethylene oxide with fatty acid alkanol amides and fatty amines, and
fatty acid N-alkyl glucamides.

Alkylene oxide addition products with saturated linear fatty alcohols and fatty acids each containing 2 to 30 mol of ethylene oxide per mol of fatty alcohol or fatty acid are preferred non-ionic surfactants. Preparations having outstanding properties are likewise obtained if they contain fatty acid esters of ethoxylated glycerol as non-ionic surfactants.

A further group of non-ionic surfactants suitable according to the invention are alkyl polyglucosides. They correspond to the formula (I)—

R¹O—[G]ₚ (1)

wherein R¹ is an alkyl and/or alkenyl residue having 4 to 22 carbon atoms, G is a sugar residue having 5 or 6 carbon atoms, and p is a number from 1 to 10. They can be obtained by relevant methods of preparative organic chemistry. Reference is made here to the documents EP-A1-301 298 and WO 90/03977 A1 as representatives of the extensive literature. Alkyl and/or alkenyl oligoglycosides can derive from aldoses or ketoses having 5 or 6 carbon atoms, preferably from glucose. Preferred alkyl and/or alkenyl oligoglycosides are thus alkyl and/or alkenyl oligoglucosides. The index value p in general formula (I) indicates the degree of oligomerization (DP) (i.e., the distribution of mono- and oligoglycosides) and is a number from 1 to 10. While p in a given compound must always be a whole number and can assume the values p=1 to 6 here, the value p for a particular alkyl oligoglycoside is a calculated quantity determined by analysis, which in most cases represents a fraction. Alkyl and/or alkenyl oligoglycosides having an average degree of oligomerization p of 1.1 to 3.0 are preferably used. From an application-oriented perspective, alkyl and/or alkenyl oligoglycosides having a degree of oligomerization below 1.7 and in particular between 1.2 and 1.7 are preferred. The alkyl or alkenyl residue R¹ can derive from primary alcohols having 4 to 11, preferably 8 to 10 carbon atoms. Typical examples are butanol, hexanol, octanol, decanol and undecanol as well as the technical mixtures thereof, such as are obtained in the hydrogenation of technical fatty acid methyl esters or during the hydrogenation of aldehydes from the Roelen oxo synthesis. Preference is given to alkyl oligoglucosides with a chain length of C₈-C₁₀ (DP=1-3) which occur as the first flush in the separation by distillation of technical C₈-C₁₈ coconut fatty alcohol and which can be contaminated with a proportion of less than 6 wt. % of C₁₋₂ alcohol, as well as to alkyl oligoglucosides based on technical C_{9/11} oxo alcohols (DP=1-3). Alkyl or alkenyl residue R¹ can further also derive from primary alcohols having 12 to 22, preferably 12 to 14 carbon atoms. Typical examples are lauryl alcohol, myristyl alcohol, cetyl alcohol, palmoleyl alcohol, stearyl alcohol, isostearyl alcohol, oleyl alcohol, elaidyl alcohol, petroselinyl alcohol, arachyl alcohol, gadoleyl alcohol, behenyl alcohol, erucyl alcohol, brassidyl alcohol as well as technical mixtures thereof such as can be obtained as described above. Most particularly preferred according to the invention are alkyl oligoglucosides based on hydrogenated C_{12/14} coconut alcohol with a DP of 1 to 3, such as are commercially available under the INCI name Coco-Glucoside.

In a particularly preferred embodiment of the invention a mixture of particularly mild surfactants is desired as a surfactant base which is suitable for use in cosmetic, conditioning skin and/or hair cleaning agents.

It is preferable according to the invention to use a mixture of mild anionic and mild amphoteric and/or zwitterionic surfactants for such a surfactant mixture. Examples of such mixtures are alkyl ether sulfates, alkyl ether carboxylic acids, acyl glutamates or sulfosuccinates, as well as alkyl betaines, sulfobetaines, alkyl amidoalkyl betaines, alkyl amphoacetates and propionates.

A mixture of alkyl ether sulfates and alkyl amidoalkyl betaines is particularly preferred. These surfactants are used in amounts of 0.1 to 45 wt. %, preferably in amounts of 1 to 30 wt. % and most particularly preferably in amounts of 1 to 17.5 wt. %, based on total weight of the agent.

In addition to the aforementioned required constituents according to the invention, the composition can also contain a series of further optional constituents. Further active ingredients having cosmetic conditioning properties are preferably added to the compositions. As such, cationic polymers, plant extracts and humectants should be mentioned in particular as further preferred optional components.

Cationic polymers suitable according to the invention refer to polymers having groups in the main and/or side chain which can be “temporarily” or “permanently” cationic. Polymers having a cationic group irrespective of the pH of the agent are described as “permanently cationic” according to the invention. These are typically polymers containing a quaternary nitrogen atom, for example, in the form of an ammonium group. Preferred cationic groups are quaternary ammonium groups. In particular, polymers wherein the quaternary ammonium group is bonded via a C₁₋₄ hydrocarbon group to a polymer main chain synthesized from acrylic acid, methacrylic acid or derivatives thereof have proved to be particularly suitable. Homopolymers of the general formula (VI)—

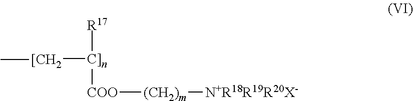

wherein R¹⁷ is —H or —CH₃, R¹⁸, R¹⁹ and R²⁰ are independently chosen from C₁₋₄ alkyl, alkenyl or hydroxyalkyl groups; m=1, 2, 3 or 4; n is a natural number; and X⁻ is a physiologically tolerable organic or inorganic anion, as well as copolymers consisting substantially of the monomer units shown in formula (III) along with non-ionogenic monomer units, are particularly preferred cationic polymers. In the context of these polymers, those for which at least one of the following conditions applies are preferred according to the invention:
R¹⁷ is a methyl group
R¹⁸, R¹⁹ and R²⁰ are methyl groups
m is 2.

Suitable physiologically tolerable counterions X⁻ include halide ions, sulfate ions, phosphate ions, methosulfate ions as well as organic ions such as lactate, citrate, tartrate and acetate ions. Halide ions, particularly chloride, are preferred.

A suitable homopolymer is the optionally crosslinked poly(methacryloyloxyethyl trimethylammonium chloride) with the INCI name Polyquaternium-37. Crosslinking can take place if desired with the aid of polyolefinically unsaturated compounds, for example divinyl benzene, tetraallyl oxyethane, methylene bisacrylamide, diallyl ether, polyallyl polyglyceryl ether, or allyl ethers of sugars or sugar derivatives such as erythritol, pentaerythritol, arabitol, mannitol, sorbitol, sucrose or glucose. Methylene bisacrylamide is a preferred crosslinking agent.

The homopolymer is preferably used in the form of a non-aqueous polymer dispersion having a polymer content of not less than 30 wt. %. Such polymer dispersions are commercially available under the names Salcare® SC 95 (approx. 50% polymer content, other components: mineral oil (INCI name: Mineral Oil) and tridecyl polyoxypropylene polyoxyethylene ether (INCI name: PPG-1-Trideceth-6)) and Salcare® SC 96 (approx. 50% polymer content, other components: mixture of diesters of propylene glycol with a mixture of caprylic and capric acid (INCI name: Propylene Glycol Dicaprylate/Dicaprate) and tridecyl polyoxypropylene polyoxyethylene ether (INCI name: PPG-1-Trideceth-6)).

Copolymers with monomer units according to formula (VI) preferably contain as non-ionogenic monomer units acrylamide, methacrylamide, acrylic acid C₁₋₄ alkyl esters and methacrylic acid C₁₋₄ alkyl esters. Of these non-ionogenic monomers acrylamide is particularly preferred. As in the case of the homopolymers described above, these copolymers too can be crosslinked. A preferred copolymer according to the invention is the crosslinked acrylamide-methacryloyloxyethyl trimethylammonium chloride copolymer. Such copolymers in which the monomers are present in a weight ratio of about 20:80 are available commercially as an approximately 50% non-aqueous polymer dispersion under the name Salcare® SC 92.

Other preferred cationic polymers include—
quaternized cellulose derivatives such as those commercially available under the names Celquat® and Polymer JR®. The compounds Celquat® H 100, Celquat® L 200 and Polymer JR®400 are preferred quaternized cellulose derivatives.
hydrophobically modified cellulose derivatives such as the cationic polymers sold under the trade name SoftCat®,
cationic alkyl polyglycosides,
cationized honey such as the commercial product Honeyquat® 50,
cationic guar derivatives such as the products sold under the trade names Cosmedia®Guar and Jaguar®,
polysiloxanes containing quaternary groups such as the commercially available products Q2-7224 (manufacturer: Dow Corning; a stabilized trimethylsilyl amodimethicone), Dow Corning® 929 Emulsion (containing a hydroxyl-amino-modified silicone, which is also known as amodimethicone), SM-2059 (manufacturer: General Electric), SLM-55067 (manufacturer: Wacker) as well as Abil®-Quat 3270 and 3272 (manufacturer: Th. Goldschmidt; diquaternary polydimethylsiloxanes, Quaternium-80),
polymeric dimethyl diallyl ammonium salts and copolymers thereof with esters and amides of acrylic acid and methacrylic acid. The products commercially available under the names Merquat®100 (poly(dimethyl diallyl ammonium chloride)) and Merquat®550 (dimethyl diallyl ammonium chloride acrylamide copolymer) are examples of such cationic polymers,
copolymers of vinyl pyrrolidone with quaternized derivatives of dialkyl aminoalkyl acrylate and methacrylate, such as for example diethyl sulfate-quaternized vinyl pyrrolidone-dimethyl aminoethyl methacrylate copolymers. Such compounds are available commercially under the names Gafquat®734 and Gafquat®755,
vinyl pyrrolidone-vinyl imidazolium methochloride copolymers as are sold under the names Luviquat® FC 370, FC 550, FC 905 and HM 552,
quaternized polyvinyl alcohol,
   as well as polymers known under the names—
Polyquaternium 2,
Polyquaternium 17,
Polyquaternium 18, and
Polyquaternium 27 having quaternary nitrogen atoms in the polymer main chain.

Polymers known under the names Polyquaternium-24 (commercially available, for example, as Quatrisoft® LM 200) can likewise be used as cationic polymers. Likewise suitable for use according to the invention are copolymers of vinyl pyrrolidone, such as are commercially available as the products Copolymer 845 (manufacturer: ISP), Gaffix® VC 713 (manufacturer: ISP), Gafquat®ASCP 1011, Gafquat® HS 110, Luviquat®8155 and Luviquat® MS 370.

In a particularly preferred embodiment of the invention at least one polymer from the group of cationic guar derivatives and/or Polyquaternium-7 (Merquat 550), Polyquaternium-6, Polyquaternium-10 and/or Polyquaternium-67 (SoftCat® polymers) is present in compositions according to the invention as a cationic polymer.

Cationic polymer(s) is (are) present in compositions according to the invention in amounts of 0.1 to 5 wt. %, based on total weight of the composition. Amounts of 0.2 to 3, particularly 0.5 to 2 wt. %, are particularly preferred.

Suitable plant extracts according to the invention refer to extracts produced from all parts of a plant. These extracts are conventionally produced by extraction of the entire plant. It can also be preferable in individual cases, however, to produce the extracts exclusively from flowers and/or leaves of the plant.

Extracts from green tea, oak bark, stinging nettle, witch hazel, hops, chamomile, burdock, horsetail, whitethorn, lime blossom, lychee, almond, aloe vera, pine, horse chestnut, sandalwood, juniper, coconut, mango, apricot, lemon, wheat, kiwi, melon, orange, grapefruit, sage, rosemary, birch, mallow, lady's smock, wild thyme, yarrow, thyme, melissa, restharrow, coltsfoot, marshmallow, ginseng, ginger root, Echinacea purpurea, Olea europaea, Foeniculum vulgaris and Apium graveolens are preferred above all according to the invention.

Water, alcohols and mixtures thereof can be used as extracting agents to produce the cited plant extracts. Of the alcohols, low alcohols such as ethanol and isopropanol, particularly polyhydric alcohols such as ethylene glycol and propylene glycol, are preferred, both as the sole extracting agent and mixed with water. Plant extracts based on water/propylene glycol in the ratio 1:10 to 10:1 have proved to be particularly suitable.

Plant extracts can be used in both pure and diluted form. If they are used in diluted form they conventionally contain approximately 2 to 80 wt. % of active substance and, as the solvent, the extracting agent or mixture of extracting agents used to obtain them.

It can also be preferable to use mixtures of a plurality of different plant extracts, particularly two.

It can additionally prove advantageous if humectants or penetration auxiliaries and/or swelling agents (M) are used in the compositions according to the invention. These auxiliary substances ensure better penetration of active ingredients into the keratin fibers or help the keratin fibers to swell. They include urea and urea derivatives, guanidine and derivatives thereof, arginine and derivatives thereof, water glass, imidazole and derivatives thereof, histidine and derivatives thereof, benzyl alcohol, glycerol, glycol and glycol ethers, propylene glycol and propylene glycol ethers, for example propylene glycol monoethyl ether, carbonates, hydrogen carbonates, diols and triols, and in particular 1,2-diols and 1,3-diols such as 1,2-propanediol, 1,2-pentanediol, 1,2-hexanediol, 1,2-dodecanediol, 1,3-propanediol, 1,6-hexanediol, 1,5-pentanediol, 1,4-butanediol. Particularly suitable according to the invention is glycerol.

Humectants are used in compositions according to the invention in amounts of 0.01 to 10 wt. %, preferably in amounts of 0.05 to 5 wt. % and in particular in amounts of 0.1 to 3 wt. %, based on total weight of the composition.

In a particularly preferred embodiment, the clear composition is packaged as a cosmetic composition for cleaning skin and/or hair, such as a hair shampoo, shower gel, shower wash, washing gel, facial cleanser, hand washing agent and/or a foam bath. As such, it can contain in addition to the aforementioned required and preferred optional components, additional active ingredients, auxiliary substances and additives which are described below.

In addition to the aforementioned required and preferred constituents, the surfactant-containing compositions can moreover contain the following components.

Likewise suitable for use according to the invention are cationic surfactants of the quaternary ammonium compound, esterquat and amidoamine type. Preferred quaternary ammonium compounds are ammonium halides, in particular chlorides and bromides, such as alkyltrimethylammonium chlorides, dialkyldimethylammonium chlorides and trialkylmethyl ammonium chlorides (e.g., cetyltrimethylammonium chloride, stearyltrimethylammonium chloride, distearyldimethylammonium chloride, lauryldimethylammonium chloride, lauryldimethylbenzylammonium chloride and tricetylmethylammonium chloride), as well as the imidazolium compounds known under the INCI names Quaternium-27 and Quatemium-83. The long alkyl chains of the aforementioned surfactants preferably have 10 to 18 carbon atoms.

Esterquats are known substances containing both at least one ester function and at least one quaternary ammonium group as a structural element. Preferred esterquats are quaternized ester salts of fatty acids with triethanolamine, quaternized ester salts of fatty acids with diethanol alkyl amines and quaternized ester salts of fatty acids with 1,2-dihydroxypropyl dialkylamines. Such products are sold under the trademarks Stepantex®, Dehyquart® and Armocare®, for example. The products Armocare® VGH-70, an N,N-bis(2-palmitoyloxyethyl)dimethylammonium chloride, as well as Dehyquart® F-75, Dehyquart® C-4046, Dehyquart® L80 and Dehyquart® AU-35 are examples of such esterquats. Alkylamidoamines are conventionally produced by amidation of natural or synthetic fatty acids and fatty acid cuts with dialkyl amino amines. A compound from this group of substances particularly suitable according to the invention is stearamidopropyl dimethylamine, commercially available under the name Tegoamid® S 18.

Cationic surfactants are preferably used in amounts of 0.05 to 10 wt. %, based on total weight of the agent. Amounts of 0.1 to 5 wt. % are particularly preferred.

In a further preferred embodiment, the action of compositions according to the invention can be increased by emulsifiers. Such emulsifiers include—
addition products of 4 to 30 mol of ethylene oxide and/or 0 to 5 mol of propylene oxide with linear fatty alcohols having 8 to 22 C atoms, with fatty acids having 12 to 22 C atoms and with alkyl phenols having 8 to 15 C atoms in the alkyl group;
C₁₂-C₂₂ fatty acid monoesters and diesters of addition products of 1 to 30 mol of ethylene oxide with polyols having 3 to 6 carbon atoms, in particular with glycerol;
ethylene oxide and polyglycerol addition products with methyl glucoside fatty acid esters, fatty acid alkanol amides and fatty acid glucamides;
C₈-C₂₂ alkyl mono- and oligoglycosides and ethoxylated analogs thereof, wherein degrees of oligomerization of 1.1 to 5, in particular 1.2 to 2.0, and glucose as the sugar component are preferred;
mixtures of alkyl (oligo)glucosides and fatty alcohols, for example the commercially available product Montanov®68;
addition products of 5 to 60 mol of ethylene oxide with castor oil and hydrogenated castor oil;
partial esters of polyols having 3 to 6 carbon atoms with saturated fatty acids having 8 to 22 C atoms;
sterols. Sterols are understood to be a group of steroids bearing a hydroxyl group on C atom 3 of the steroid skeleton and are isolated from both animal tissue (zoosterols) and vegetable fats (phytosterols). Examples of zoosterols are cholesterol and lanosterol. Examples of suitable phytosterols are ergosterol, stigmasterol and sitosterol. Sterols known as mycosterols are also isolated from fungi and yeasts.
phospholipids. These are understood above all to be the glucose phospholipids which are obtained for example as lecithins or phosphatidyl cholines from for example egg yolk or plant seeds (e.g., soybeans).
fatty acid esters of sugars and sugar alcohols such as sorbitol;
polyglycerols and polyglycerol derivatives such as polyglycerol poly-12-hydroxystearate (commercial product Dehymuls® PGPH);
linear and branched fatty acids having 8 to 30 C atoms and Na, K, ammonium, Ca, Mg and Zn salts thereof.

Emulsifiers are preferably used in amounts of 0.1 to 25 wt. %, particularly 0.5 to 15 wt. %, based on total weight of the agent.

It can moreover be advantageous if, in addition to cationic polymers, compositions according to the invention further contain natural and/or synthetic polymers which are anionically or amphoterically charged as well as non-ionic.

Anionic polymers are conventionally an anionic polymer having carboxylate and/or sulfonate groups. Examples of anionic monomers which can constitute such polymers are acrylic acid, methacrylic acid, crotonic acid, maleic anhydride and 2-acrylamido-2-methylpropane sulfonic acid. Some or all of the acid groups therein can be present as the sodium, potassium, ammonium, mono- or triethanolammonium salt. Preferred monomers are 2-acrylamido-2-methylpropane sulfonic acid and acrylic acid.

Anionic polymers containing as the sole monomer or as a co-monomer 2-acrylamido-2-methylpropane sulfonic acid, in which some or all of the sulfonic acid group can be present as the sodium, potassium, ammonium, mono- or triethanolammonium salt, have proved to be most particularly effective.

The homopolymer of 2-acrylamido-2-methylpropane sulfonic acid, commercially available, for example, under the name Rheothik® 11-80, is particularly preferred.

Within this embodiment it can be preferable to use copolyrtiers consisting of at least one anionic monomer and at least one non-ionogenic monomer. Reference is made to the aforementioned substances with regard to the anionic monomers. Preferred non-ionogenic monomers are acrylamide, methacrylamide, acrylic acid ester, methacrylic acid ester, vinyl pyrrolidone, vinyl ether and vinyl ester.

Preferred anionic copolymers are acrylic acid-acrylamide copolymers as well as in particular polyacrylamide copolymers with monomers containing sulfonic acid groups. A particularly preferred anionic copolymer consists of 70 to 55 mol % of acrylamide and 30 to 45 mol % of 2-acrylamido-2-methylpropane sulfonic acid, wherein some or all of the sulfonic acid group is present as the sodium, potassium, ammonium, mono- or triethanolammonium salt. This copolymer can also be crosslinked, wherein polyolefinically unsaturated compounds such as tetraallyl oxyethane, allyl sucrose, allyl pentaerythritol and methylene bisacrylamide are preferably used as crosslinking agents. Such a polymer is present in the commercial product Sepigel®305 from SEPPIC. Use of this compound, which in addition to the polymer component contains a mixture of hydrocarbons (C₁₃-C₁₄ isoparaffin) and a non-ionogenic emulsifier (Laureth-7), has proved to be particularly advantageous within the context of the teaching according to the invention.

Sodium acryloyl dimethyl taurate copolymers sold under the name Simulgel®600 as a compound with isohexadecane and Polysorbate-80 have also proved to be particularly effective according to the invention.

Likewise preferred anionic homopolymers are uncrosslinked and crosslinked polyacrylic acids. Allyl ethers of pentaerythritol, of sucrose and of propylene can be preferred crosslinking agents here. Such compounds are available commercially for example under the trademark Carbopol®.

Copolymers of maleic anhydride and methyl vinyl ether, in particular those with crosslinkages, are likewise color-retaining polymers. A maleic acid-methyl vinyl ether copolymer crosslinked with 1,9-decadiene is available commercially under the name Stabileze® QM.

Amphoteric polymers can also be used as a polymer constituent to increase the action of the agent according to the invention. The term amphoteric polymers encompasses both polymers having both free amino groups and free —COOH or SO₃H groups in the molecule and which are capable of forming internal salts, and zwitterionic polymers having quaternary ammonium groups and —COO⁻ or —SO₃⁻ groups in the molecule, and such polymers containing —COOH or SO₃H groups and quaternary ammonium groups.

An example of an amphopolymer which can be used according to the invention is the acrylic resin available under the name Amphomer®, which is a copolymer of tert-butyl aminoethyl methacrylate, N-(1,1,3,3-tetramethylbutyl)acrylamide and two or more monomers from the group acrylic acid, methacrylic acid and simple esters thereof.

Preferred amphoteric polymers are polymers composed substantially of—

(a) monomers having quaternary ammonium groups of the general formula (PII),

R²²—CH═CR²³—CO—Z—(CₙH₂ₙ)—N⁽⁺⁾R²⁴R²⁵R²⁶A⁽⁻⁾ (PII)
wherein R²² and R²³ are each independently hydrogen or a methyl group; R²⁴, R²⁵ and R²⁶ are each independently alkyl groups having 1 to 4 carbon atoms; Z is an NH group or an oxygen atom; n is a whole number from 2 to 5; and A⁽⁻⁾ is the anion of an organic or inorganic acid, and
   (b) monomeric carboxylic acids of the general formula (PIII),

R²⁷—CH═CR²⁸—COOH (PIII)
wherein R²⁷ and R²⁸ are each independently hydrogen or methyl groups.
These compounds can be used according to the invention both directly and in the form of their salt, obtained by neutralization of the polymer, with an alkali hydroxide, for example. Regarding production of these polymers, reference is made to the content of German Patent Application No. 39 29 973. Polymers in which monomers of type (a) are used, wherein R²⁴, R²⁵ and R²⁶ are methyl groups, Z is an NH group and A⁽⁻⁾ is a halide, methoxy sulfate or ethoxy sulfate ion, are most particularly preferred; acrylamidopropyl trimethylammonium chloride is a particularly preferred monomer (a). Acrylic acid is preferably used as the monomer (b) for the cited polymers.

Suitable non-ionogenic polymers according to the invention include—
vinyl pyrrolidone/vinyl ester copolymers, such as are sold under the trademark Luviskol®(BASF). Luviskol® VA 64 and Luviskol® VA 73, both of which are vinyl pyrrolidone/vinyl acetate copolymers, are likewise preferred non-ionic polymers;
cellulose ethers such as hydroxypropyl cellulose, hydroxyethyl cellulose and methylhydroxypropyl cellulose, such as are sold under the trademarks Culminal® and Benecel® (AQUALON);
shellac;
polyvinyl pyrrolidones, such as are sold for example under the name Luviskol® (BASF);
siloxanes. These siloxanes can be both water-soluble and water-insoluble. Both volatile and non-volatile siloxanes are suitable, wherein compounds whose boiling point under normal pressure is above 200° C. are understood to be non-volatile siloxanes. Preferred siloxanes are polydialkyl siloxanes, such as for example polydimethyl siloxane, polyalkylaryl siloxanes, such as for example polyphenylmethyl siloxane, ethoxylated polydialkyl siloxanes as well as polydialkyl siloxanes containing amine and/or hydroxyl groups; and
glycoside-substituted silicones.

Use of the further polymers in amounts of 0.01 to 10 wt. %, based on total weight of the agent, is preferred according to the invention. Amounts of 0.1 to 5 wt. %, particularly 0.1 to 3 wt. %, are even more preferred.

In a further embodiment, protein hydrolysates and/or derivatives thereof can be used to increase the action of the composition according to the invention. Protein hydrolysates are mixtures of products obtained by acidically, basically or enzymatically catalyzed breakdown of proteins of both plant and animal origin.

Animal protein hydrolysates include elastin, collagen, keratin, silk and milk protein hydrolysates, which can also be present in the form of salts. Such products are available under the trademarks Dehylan® (Cognis), Promois® (Interorgana), Collapuron® (Cognis), Nutrilan® (Cognis), Gelita-Sol® (Deutsche Gelatine Fabriken Stoess & Co), Lexein® (Inolex) and Kerasol® (Croda).

Preferred according to the invention are protein hydrolysates of plant origin such as soy, almond, rice, pea, potato and wheat protein hydrolysates. Such products are available for example under the trademarks Gluadin® (Cognis), DiaMin® (Diamalt), Lexein® (Inolex) and Crotein® (Croda).

Although use of protein hydrolysates as such is preferred, other amino acid mixtures obtained by other means or individual amino acids such as arginine, lysine, histidine or pyroglutamic acid can optionally be used in their place. Use of derivatives of protein hydrolysates, for example, in the form of their fatty acid condensation products, is likewise possible. Such products are available under the names Lamepon® (Cognis), Gluadin® (Cognis), Lexein® (Inolex), Crolastin® (Croda) or Crotein® (Croda).

Also suitable for use according to the invention are cationized protein hydrolysates, wherein the underlying protein hydrolysate can derive from animal sources, for example, from collagen, milk or keratin, from plant sources, for example from wheat, maize, rice, potatoes, soy or almonds, from marine life forms, for example from fish collagen or algae, or from protein hydrolysates obtained by biotechnology. Protein hydrolysates underlying the cationic derivatives according to the invention can be obtained from the corresponding proteins by chemical, in particular alkali or acid hydrolysis, enzymatic hydrolysis, or a combination of both types of hydrolysis. Cationic protein hydrolysates are also understood to include quaternized amino acids and mixtures thereof. Quaternization of protein hydrolysates or amino acids is frequently performed using quaternary ammonium salts such as N,N-dimethyl-N-(n-alkyl)-N-(2-hydroxy-3-chloro-n-propyl) ammonium halides. Cationic protein hydrolysates can also be further derivatized. Typical examples of cationic protein hydrolysates and derivatives according to the invention are products listed under the INCI names in the “International Cosmetic Ingredient Dictionary and Handbook”, (7^{th} Edition (1997), The Cosmetic, Toiletry, and Fragrance Association, 1101 17th Street, N.W., Suite 300, Washington, D.C. 20036-4702) and which are available commercially: Cocodimonium Hydroxypropyl Hydrolyzed Collagen, Cocodimonium Hydroxypropyl Hydrolyzed Casein, Cocodimonium Hydroxypropyl Hydrolyzed Collagen, Cocodimonium Hydroxypropyl Hydrolyzed Hair Keratin, Cocodimonium Hydroxypropyl Hydrolyzed Keratin, Cocodimonium Hydroxypropyl Hydrolyzed Rice Protein, Cocodimonium Hydroxypropyl Hydrolyzed Silk, Cocodimonium Hydroxypropyl Hydrolyzed Soy Protein, Cocodimonium Hydroxypropyl Hydrolyzed Wheat Protein, Cocodimonium Hydroxypropyl Silk Amino Acids, Hydroxypropyl Arginine Lauryl/Myristyl Ether HCl, Hydroxypropyltrimonium Gelatin, Hydroxypropyltrimonium Hydrolyzed Casein, Hydroxypropyltrimonium Hydrolyzed Collagen, Hydroxypropyltrimonium Hydrolyzed Conchiolin Protein, Hydroxypropyltrimonium Hydrolyzed Keratin, Hydroxypropyltrimonium Hydrolyzed Rice Bran Protein, Hydroxyproypltrimonium Hydrolyzed Silk, Hydroxypropyltrimonium Hydrolyzed Soy Protein, Hydroxypropyl Hydrolyzed Vegetable Protein, Hydroxypropyltrimonium Hydrolyzed Wheat Protein, Hydroxypropyltrimonium Hydrolyzed Wheat Protein/Siloxysilicate, Laurdimonium Hydroxypropyl Hydrolyzed Soy Protein, Laurdimonium Hydroxypropyl Hydrolyzed Wheat Protein, Laurdimonium Hydroxypropyl Hydrolyzed Wheat Protein/Siloxysilicate, Lauryldimonium Hydroxypropyl Hydrolyzed Casein, Lauryldimonium Hydroxypropyl Hydrolyzed Collagen, Lauryldimonium Hydroxypropyl Hydrolyzed Keratin, Lauryldimonium Hydroxypropyl Hydrolyzed Silk, Lauryldimonium Hydroxypropyl Hydrolyzed Soy Protein, Steardimonium Hydroxypropyl Hydrolyzed Casein, Steardimonium Hydroxypropyl Hydrolyzed Collagen, Steardimonium Hydroxypropyl Hydrolyzed Keratin, Steardimonium Hydroxypropyl Hydrolyzed Rice Protein, Steardimonium Hydroxypropyl Hydrolyzed Silk, Steardimonium Hydroxypropyl Hydrolyzed Soy Protein, Steardimonium Hydroxypropyl Hydrolyzed Vegetable Protein, Steardimonium Hydroxypropyl Hydrolyzed Wheat Protein, Steartrimonium Hydroxyethyl Hydrolyzed Collagen, Quaternium-76 Hydrolyzed Collagen, Quaternium-79 Hydrolyzed Collagen, Quaternium-79 Hydrolyzed Keratin, Quaternium-79 Hydrolyzed Milk Protein, Quaternium-79 Hydrolyzed Silk, Quaternium-79 Hydrolyzed Soy Protein, Quaternium-79 Hydrolyzed Wheat Protein. Plant-based cationic protein hydrolysates and derivatives are most particularly preferred.

Protein hydrolysates and derivatives thereof are preferably used in amounts of 0.01 to 10 wt. %, based on total weight of the agent. Amounts of 0.1 to 5 wt. %, particularly 0.1 to 3 wt. %, are most particularly preferred.

The combination of the composition according to the invention with vitamins, provitamins and vitamin precursors as well as derivatives thereof has likewise proved advantageous.

Vitamins, provitamins and vitamin precursors preferred according to the invention are conventionally assigned to groups A, B, C, E, F and H.

Substances classified as vitamin A includes retinol (vitamin A₁) and 3,4-didehydroretinol (vitamin A₂). β-Carotene is the retinol provitamin. Suitable vitamin A components according to the invention include vitamin A acid and esters thereof, vitamin A aldehyde and vitamin A alcohol and esters thereof such as palmitate and acetate. The vitamin A component is preferably used in amounts of 0.05 to 1 wt. %, based on total weight of the preparation.

The vitamin B group or vitamin B complex includes, inter alia—
vitamin B₁ (thiamine)
vitamin B₂ (riboflavin)
vitamin B₃. The compounds nicotinic acid and nicotinic acid amide (niacinamide) are often included under this term. Preferred according to the invention is nicotinic acid amide, which is preferably used in amounts of 0.05 to 1 wt. %, relative to the complete agent.
vitamin B₅ (pantothenic acid and panthenol). Within the context of this group panthenol is preferably used. Derivatives of panthenol which can be used according to the invention are in particular the esters and ethers of panthenol as well as cationically derivatized panthenols. Individual representatives are for example panthenol triacetate, panthenol monoethyl ether and the monoacetate thereof as well as the cationic panthenol derivatives disclosed in WO 92/13829. The cited compounds of the vitamin B₅ type are preferably used in amounts of 0.05 to 10 wt. %, based on total weight of the agent. Amounts of 0.1 to 5 wt. % are particularly preferred.
vitamin B₆ (pyridoxine as well as pyridoxamine and pyridoxal).

Vitamin C (ascorbic acid). The amount of vitamin C conventionally used is 0.1 to 3 wt. %, based on total weight of the agent. Use in the form of the palmitic acid ester, glucosides or phosphates can be preferred. Use in combination with tocopherols can likewise be preferred.

Vitamin E (tocopherols, in particular α-tocopherol). Tocopherol and derivatives thereof, which include in particular the esters such as acetate, nicotinate, phosphate and succinate, are preferably used according to the invention in amounts of 0.05 to 1 wt. %, based on total weight of the agent.

Vitamin F. The term “vitamin F” is conventionally understood to mean essential fatty acids, in particular linoleic acid, linolenic acid and arachidonic acid.

Vitamin H. Vitamin H is the name given to the compound (3aS,4S,6aR)-2-oxohexahydrothienol[3,4-d]-imidazole-4-valeric acid, although this is now more widely known by the trivial name biotin. Biotin is preferably used in amounts of 0.0001 to 1.0 wt. %, in particular in amounts of 0.001 to 0.01 wt. %, based on total weight of the agent.

Particularly preferred according to the invention is the use of vitamins, provitamins and vitamin precursors from groups A, B, E and H.

Panthenol and derivatives thereof as well as nicotinic acid amide and biotin are particularly preferred.

A UV filter (I) can also be used in a preferred embodiment of the invention. There are no general restrictions on UV filters used according to the invention in terms of their structure and physical properties. In fact, all UV filters useful in the cosmetics sector whose absorption maximum is in the UVA (315-400 nm), UVB (280-315 nm) or UVC 280 nm) range are suitable. UV filters having an absorption maximum in the UVB range, particularly in the range from approximately 280 to approximately 300 nm, are particularly preferred.

UV filters can be chosen from substituted benzophenones, p-aminobenzoic acid esters, diphenyl acrylic acid esters, cinnamic acid esters, salicylic acid esters, benzimidazoles and o-aminobenzoic acid esters.

According to a further embodiment of the invention UV filters having a cationic group, in particular a quaternary ammonium group, are preferred.

Two preferred UV filters having cationic groups are the compounds cinnamic acid amidopropyl trimethylammonium chloride (Incroquat®UV-283) and dodecyl dimethylaminobenzamidopropyl dimethylammonium tosylate (Escalol® HP 610), available as commercial products. The teaching according to the invention also includes the combination of a plurality of UV filters.

UV filters (I) are conventionally used in amounts of 0.1 to 5 wt. %, based on total weight of the agent. Amounts of 0.4 to 2.5 wt. % are preferred.

There are no restrictions in principle regarding the nature of the cosmetic preparations according to the invention. Creams, lotions, solutions, waters, emulsions such as W/O, O/W, PIT emulsions (emulsions based on phase inversion teaching, known as PIT), microemulsions and multiple emulsions, coarse, unstable, single-phase or multi-phase shaking mixtures, gels, sprays, aerosols and foam aerosols, for example, are suitable as a packaging for these preparations.

Other active ingredients, auxiliary substances and additives include—
thickening agents such as gelatins or plant gums, for example agar-agar, guar gum, alginates, xanthan gum, gum arabic, karaya gum, carob seed meal, linseed gums, dextrans, cellulose derivatives, for example, methyl cellulose, hydroxyalkyl cellulose and carboxymethyl cellulose, starch fractions and derivatives such as amylose, amylopectin and dextrins, clays and layered silicates such as bentonite or fully synthetic hydrocolloids such as polyvinyl alcohol, the Ca, Mg or Zn soaps,
texturizing agents such as maleic acid and lactic acid,
dimethyl isosorbide,
cyclodextrins,
solvents and solubilizers such as ethanol, isopropanol, ethylene glycol, propylene glycol, glycerol and diethylene glycol,
active ingredients to improve the fiber structure, in particular mono-, di- and oligosaccharides such as glucose, galactose, fructose, fruit sugar and lactose,
quaternized amines such as methyl-1-alkylamidoethyl-2-alkylimidazolinium methosulfate,
dyes to color the agent,
anti-dandruff active ingredients such as piroctone olamine, zinc omadine and climbazole,
further substances to adjust the pH, such as α- and β-hydroxycarboxylic acids,
active ingredients such as allantoin and bisabolol,
cholesterol,
complexing agents such as EDTA, NTA, β-alanine diacetic acid and phosphonic acids,
ceramides. Ceramides are understood to be N-acyl sphingosine (fatty acid amides of sphingosine) or synthetic analogs of such lipids (known as pseudoceramides),
opacifiers such as latex, styrene/PVP and styrene/acrylamide copolymers,
pearlescent agents such as ethylene glycol mono- and distearate as well as PEG-3 distearate,
pigments,
reducing agents such as thioglycolic acid and derivatives thereof, thiolactic acid, cysteamine, thiomalic acid and α-mercaptoethane sulfonic acid,
blowing agents such as propane-butane mixtures, N₂O, dimethyl ether, CO₂ and air,
antioxidants,
deoxy sugars, plant glycosides, polysaccharides such as fucose or rhamnose,
consistency modifiers such as sugar esters, polyol esters or polyol alkyl ethers,
fats and waxes such as spermaceti wax, beeswax, montan wax and paraffins,
fatty acid alkanol amides,
hydrogen carbonates, guanidines, ureas as well as primary, secondary and tertiary phosphates,
preservatives such as sodium benzoate,
viscosity adjusters such as salts (NaCl).

A second subject-matter of the invention is the use of a combination of solubilizers chosen from—
a) ethoxylated fatty alcohols,
b) ethoxylated hydrogenated castor oils, and
c) ethoxylated mono-, di- or triglycerol esters,
   in a ratio of components a):b):c) in the range of 1:(2-4):(3-4) to suspend and stabilize an oil component in a clear composition containing surfactants and to produce a composition having a stable, constant viscosity in the range from 5000 to 9500 mPas.

The following examples illustrate the invention without however restricting its scope. Unless otherwise specified, all figures are percentages by weight.

### EXAMPLES

| Raw material | 1 | 2 | 3 | 4 | 5 | 6 | 7 | 8 | 9 | 10 | 11 | 12 | 13 | 14 |
|---|---|---|---|---|---|---|---|---|---|---|---|---|---|---|
| (SLES), 2 EO, | 10 | 10 | 10 | 10 | 10 | 10 | 10 | 10 | 10 | 10 | 10 | 10 | 10 | 10 |
| 70% | | | | | | | | | | | | | | |
| CAPB 40% | 10 | 10 | 10 | 10 | 10 | 10 | 10 | 10 | 10 | 10 | 10 | 10 | 10 | 10 |
| Conditioner ®¹ | 1.5 | 1.5 | 1.5 | 1.5 | 1.5 | 1.5 | 1.5 | 1.5 | 1.5 | 1.5 | 1.5 | 1.5 | 1.5 | 1.5 |
| P7 | | | | | | | | | | | | | | |
| Glycerol 86% | 1 | 1 | 1 | 1 | 1 | 1 | 1 | 1 | 1 | 1 | 1 | 1 | 1 | 1 |
| Herbasol | 0.1 | 0.1 | 0.1 | 0.1 | 0.1 | 0.1 | 0.1 | 0.1 | 0.1 | 0.1 | 0.1 | 0.1 | 0.1 | 0.1 |
| extract, lychee | | | | | | | | | | | | | | |
| Sodium | 0.4 | 0.4 | 0.4 | 0.4 | 0.4 | 0.4 | 0.4 | 0.4 | 0.4 | 0.4 | 0.4 | 0.4 | 0.4 | 0.4 |
| benzoate | | | | | | | | | | | | | | |
| Citric acid | 0.2 | 0.2 | 0.2 | 0.2 | 0.2 | 0.2 | 0.2 | 0.2 | 0.2 | 0.2 | 0.2 | 0.2 | 0.2 | 0.2 |
| Almond oil, | 0.2 | 0.2 | 0.2 | 0.2 | 0.2 | 0.2 | 0.2 | 0.2 | 0.2 | 0.2 | 0.2 | 0.2 | 0.2 | 0.2 |
| sweet | | | | | | | | | | | | | | |
| Perfume | 1 | 1 | 1 | 1 | 1 | 1 | 1 | 1 | 1 | 1 | 1 | 1 | 1 | 1 |
| Solubilizer | | 1 | 1 | 1 | | | | | | | 1 | 0.5 | 0.5 | 0.5 |
| 660352 ®² | | | | | | | | | | | | | | |
| Cetiol ®³ HE | 1 | | 1 | 1 | 1 | 1 | 0.5 | 0.7 | 1 | 1 | 1 | 1 | 1 | 0.8 |
| Eumulgin ®⁴ | 0.3 | | | 0.3 | 0.2 | 0.5 | | | | | 0.2 | 0.2 | 0.4 | 0.4 |
| HRE 455 | | | | | | | | | | | | | | |
| Eumulgin ®⁵ | | | | | | | 0.3 | 0.3 | 0.2 | 0.3 | | | | |
| HRE 40 | | | | | | | | | | | | | | |
| NaCI | 0.9 | 0.9 | 0.2 | | 1 | 1 | | 1 | 1.2 | 1.2 | 0.2 | 0.2 | 0.2 | 0.4 |
| Antil ®⁶ 141 | 0.2 | 0.2 | 0.6 | 1 | | | | | 0.5 | 0.5 | 0.5 | 0.5 | 0.5 | 0.6 |
| Arlypon ®⁷ F | 0.2 | 0.2 | | | 0.5 | | | | | | | | | 0.6 |
| Water | to 100 | to 100 | to 100 | to 100 | to 100 | to 100 | to 100 | to 100 | to 100 | to 100 | to 100 | to 100 | to 100 | to 100 |
| Viscosity in | 3.2 | 2.6 | | | | | | | 7.4 | 6.8 | | | | 7.9 |
| Pas | | | | | | | | | | | | | | |
| Appearance | c | c | t | t | st | c | t | t | c | c | t | st | c | c |
| 3 days (stored | c | st | | | | c | | | t | t | | | | c |
| at 60° C.) | | | | | | | | | | | | | | |
| 7 days (stored | | | | | | | | | t | t | | | | c |
| at 50° C.) | | | | | | | | | | | | | | |
| 3 days (room | c | st | | | | c | | | | | | | | |
| temp.) | | | | | | | | | | | | | | |
| >3 days | t | | | | | | | | | | t | st | st | c |

t = turbid;
c = clear;
st = slightly turbid

It can be seen from column 14 of the table that only compositions according to the invention meet all requirements in terms of viscosity stability and suspension stability.

The following commercial products were used—
SLES: INCI name: Sodium Laureth Sulfate (2 EO); 68-75% AS; Cognis
CAPB: INCI name: Cocamidopropyl Betaine; approx. 44% AS; Cognis
1 INCI name: Polyquaternium-7, Aqua; Sigma
2 INCI name: PEG-40 Hydrogenated Castor Oil, Trideceth-9, Propylene Glycol, Aqua; Symrise
3 INCI name: PEG-7 Glyceryl Cocoate; Cognis
4 INCI name: PEG-40 Hydrogenated Castor Oil, Propylene Glycol, Aqua; Cognis
5 INCI name: PEG-40 Hydrogenated Castor Oil; Cognis
6 INCI name: Propylene Glycol, PEG-55 Propylene Glycol Oleate; Goldschmidt
7 INCI name: Laureth-2; Cognis

We claim:

1. Clear composition comprising:
one or more surfactants,
one or more oil components, and
a mixture of solubilizers chosen from
a) ethoxylated fatty alcohols,
b) ethoxylated hydrogenated castor oils, and
c) ethoxylated mono-, di- or triglycerol esters,
wherein the ratio of components a):b):c) is in the range of 1:(2-4):(3-4).

2. Composition according to claim 1, wherein it has an NTU value (nephelometric turbidity unit) of ≦50.

3. Composition according to claim 1, wherein component a) is a straight-chain or branched, saturated or unsaturated C₈-C₂₂ fatty alcohols having a degree of ethoxylation of 1 to 18.

4. Composition according to claim 1, wherein component b) has a degree of ethoxylation of 5 to 80.

5. Composition according to claim 1, wherein component c) is chosen from monoesters and/or mixtures of monoesters and diesters of glycerol with branched or straight-chain, saturated or unsaturated fatty acids having a C chain length of 8 to 24 and a degree of ethoxylation of 1 to 20.

6. Composition according to claim 1 comprising components a), b) and c) each in amounts of 0.05 to 3 wt %, based on total weight of the composition.

7. Composition according to claim 1, wherein it has a viscosity in a range of from 5000 to 9500 mPas (as measured using a Haake rotary viscometer VT550; 20° C.; measuring device MV; spindle MV II; 8 rpm).

8. Composition according to claim 1, wherein the one or more oil components are chosen from mineral, natural or synthetic oil components present in an amount of 0.005 to 20 wt. %, based on total weight of the composition.

9. Composition according to claim 8, wherein the one or more oil components are natural plant oils.

10. Composition according to claim 1 wherein the one or more surfactants is at least a mixture of mild surfactants comprising at least one anionic and at least one amphoteric/zwitterionic surfactant.

11. Composition according to claim 1 further comprising at least one active ingredient chosen from cationic polymers, plant extracts and humectants.

12. Composition according to claim 1, wherein it is a cosmetic composition for cleaning the skin and/or hair.

13. Composition according to claim 12, wherein the cosmetic composition is a hair shampoo, shower gel, shower wash, washing gel, facial cleanser, hand washing agent and/or a foam bath.
